(19) United States
(12) Patent Application Publication
(10) Pub. No.: US 2021/0332073 A1
(43) Pub. Date: Oct. 28, 2021

(54) PROCESS FOR THE PREPARATION OF FERRIC CITRATE

(71) Applicant: RK Pharma Solutions LLC, Piscataway, NJ (US)

(72) Inventors: Ravishanker Kovi, Monroe Township, NJ (US); Jayaraman Kannappan, Vadodara (IN); Shivnath Patil, Taluka District-Dhule (IN); Srikrishna Apar, Maharashtra (IN); Govind Ahirrao, Jalgaon (IN); Shrikant Wadajkar, Pune (IN)

(21) Appl. No.: 17/034,030

(22) Filed: Sep. 28, 2020

(30) Foreign Application Priority Data
Apr. 26, 2020 (IN) 202021017797

(51) Int. Cl. C07F 15/02

(52) U.S. Cl. CPC C07F 15/025

(57) ABSTRACT

Method are provided for obtaining ferric citrate that includes the step(s) of reacting ferric chloride with a suitable base to obtain ferric hydroxide; reacting the ferric hydroxide with citric acid to obtain ferric citrate; and processing the ferric citrate to obtain ferric citrate. The resulting ferric citrate has a specific surface area greater than 16 sq. m/g, but less than 20 sq. m/g.

### FIELD OF THE INVENTION

The present application relates to ferric citrate of formula I. Specifically, the present application relates to improved process(es) for the preparation of ferric citrate, pharmaceutical compositions containing ferric citrate, and the use of this ferric citrate composition in the treatment of hyperphosphatemia and metabolic acidosis.

### BACKGROUND

Ferric citrate (also called AURYXIA®) is a phosphate binder and iron replacement product, which is known chemically as iron (+3), x (1,2,3-propanetricarboxylic acid, 2-hydroxy-), y (H₂O). Ferric citrate has an empirical formula of FeC₆H₅O₇ and a formula weight of 244.9 gm/mol. The structure of ferric citrate is depicted in formula I.

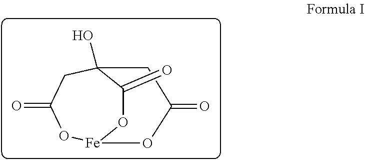

Auryxia® 210 mg ferric iron tablets are for oral administration, equivalent to lg ferric citrate, are film-coated, peach-colored, and oval-shaped tablets debossed with “KX52”. The inactive ingredients are pregelatinized starch and calcium stearate. In addition, the film-coating contains the following inactive ingredients: hypromellose, titanium dioxide, triacetin, and FD&C Yellow #6/Sunset Yellow FCF Aluminum Lake, FD&C Red #40/Allura Red AC Aluminum Lake, and FD&C Blue #2/Indigo Carmine Aluminum Lake.

Ferric citrate is garnet-red, transparent scales or pale-brown powder, odorless and slightly ferruginous tasting. Ferric citrate is slowly but completely soluble in cold water and readily soluble in hot water but diminishes in solubility with age, as disclosed in Merck Index.

Ferric iron containing compounds are useful in the treatment of several disorders and are approved for use in various treatments. In particular, use of ferric citrate includes hyperphosphatemia and metabolic acidosis. Hyperphosphatemia is associated with severe complications, such as hypocalcemia, decreasing of vitamin-D production, metastatic calcification. Hyperphosphatemia is also contributing to the increased incidence of cardiovascular disease among dialysis-dependent patients and can result in bone pathology. Specifically, Auryxia® is indicated for the control of serum phosphorus levels in adult patients with chronic kidney disease on dialysis. In addition, Auryxia® is also indicated for the treatment of iron deficiency anemia in adult patients with chronic kidney disease not on dialysis.

Early literature discussing the synthesis of ferric citrate includes U.S. Pat. No. 6,903,235, which is incorporated herein by reference, discusses the process for the preparation of pharmaceutical grade ferric citrate in solid phase. PCT Publication No. WO/2004/074444, which is also incorporated herein by reference, discusses a method of preparing ferric organic compounds, such as ferric citrate that purportedly remains soluble over a wider range of pH than previously described preparations and that has a relatively large active surface area. This process incorporates isolation of ferric hydroxide to get ferric citrate. This reference, however, does not provide commercially scalable manufacturing processes with quality control/analysis measures to ensure and/or to verify the compliance of the pharmaceutical-grade ferric citrate or ferric organic compounds with established standards or specifications.

PCT Publication No. WO/2007/022435, which is also incorporated herein by reference, is a continuation-in-part of WO/2004/074444, further discusses the process for the preparation of ferric citrate having specific surface area above 16 sq. m/g, but attempts to replicate this result regarding specific surface area, the results were not consistent. Another reference, Publication No. WO/2017/021921, which is also incorporated herein by reference, discusses the process for preparation of ferric citrate using a one pot process.

While the processes in these references purportedly provide ferric citrate, some with the desired surface area, the processes discussed therein do not provide the desired results consistently. Accordingly, there is a need improved, cost effective, and/or plant scalable processes for the preparation of pharmaceutical grade ferric citrate having a specific surface area consistently, e.g., in a consistent range.

### SUMMARY

In general, the present application provides improved process(es) for the synthesis of ferric citrate of formula I. Ferric citrate obtained through the process(es) disclosed herein preferably have a specific surface area greater than 16 sq. m/g.

In one aspect of present application provides process(es) for the preparation of crude ferric citrate.

In a preferred embodiment, this process involves the step(s) of: a) Substitution reaction of ferric chloride of formula IV with a suitable base to get ferric hydroxide of formula III, and b) Reaction of ferric hydroxide with citric acid of formula II to get Ferric citrate of formula I. A schematic representation of the improved process according to the process disclosed herein is depicted in FIG. 1.

In another aspect, the present application provides improved process(es) for the preparation of ferric citrate having specific surface area greater than 16 sq. m/g. Several processes are provided to meet this requirement.

In one embodiment, the process involves a spraying of water onto ferric citrate having a specific surface area greater than 20 sq. m/g, followed by shifting, blending and drying to get ferric citrate having a reduced specific surface area that is greater than 16 sq. m/g.

In another embodiment, the process involves charging a ferric citrate having a specific surface area greater than 20 sq. m/g onto mixture of water and solvent to get ferric citrate having a reduced specific surface area that is greater than 16 sq. m/g.

In yet another embodiment, the process involves dividing a crude filtrate ml in two equal parts. Suitable solvent is then charged/dumped into individual lots to get ferric citrate having a specific surface area in particular range. Both lots may then be mixed by solid blending or chemical mixing in particular solvent or mixture of solvents to get ferric citrate having a specific surface area greater than 16 sq. m/g.

In one aspect, a method is provided for obtaining ferric citrate that includes the step(s) of reacting ferric chloride with a suitable base to obtain ferric hydroxide; reacting the ferric hydroxide with citric acid to obtain ferric citrate; and processing the ferric citrate to obtain ferric citrate having a specific surface area greater than 16 sq. m/g, but less than 20 sq. m/g.

In at least one embodiment, the ferric chloride is reacted with a suitable base in the presence of water, and wherein the suitable base comprises one or more of: sodium hydroxide, potassium hydroxide, lithium hydroxide, calcium hydroxide, caesium hydroxide, potassium carbonate, sodium carbonate, potassium bicarbonate, and sodium bicarbonate.

In at least one embodiment, the ferric chloride is ferric chloride formula IV.

In at least one embodiment, the ferric hydroxide is ferric hydroxide formula III.

In at least one embodiment, the citric acid is citric acid formula II.

In at least one embodiment, the ferric citrate is ferric citrate formula I.

In at least one embodiment, the ferric citrate obtained is in liquid form.

In at least one embodiment, processing the ferric citrate comprises filtering the liquid ferric citrate to obtain liquid filtrate ml of ferric citrate.

In at least one embodiment, the method includes isolating ferric citrate from the liquid filtrate ml of ferric citrate.

In at least one embodiment, isolating ferric citrate comprises adding a suitable solvent to the liquid filtrate ml of ferric citrate, slurring resulting solid material in the solvent, and drying the solid material.

In at least one embodiment, isolating ferric citrate further comprises passing dried ferric citrate through a sieve to obtain ferric citrate powder having surface area greater than 20 sq. m/g.

In at least one embodiment, isolating the ferric citrate further comprises spraying the ferric citrate powder with water, blending the mixture, drying the mixture, and passing the dried mixture through a sieve to obtain the ferric citrate having a specific surface area greater than 16 sq. m/g, but less than 20 sq. m/g.

In at least one embodiment, isolating the ferric citrate further comprises charging the dried ferric citrate into a solvent, blending, the mixture, drying the mixture, and passing the dried mixture through a sieve to obtain the ferric citrate having a specific surface area greater than 16 sq. m/g, but less than 20 sq. m/g.

In at least one embodiment, isolating the ferric citrate further comprises charging the ferric citrate powder with a solvent, stirring the reaction mass, and washing the reaction mass with the solvent.

In at least one embodiment, the solvent comprises at least one of: acetone, methanol, isopropyl alcohol and tetrahydrofuran.

In at least one embodiment, isolating the ferric citrate further comprises dividing the liquid filtrate ml of ferric citrate into at least two equal lots; charging each lot of filtrate with a suitable solvent, mixing each lot, creating a slurry comprising the at least two lots, drying the combined lots, and passing the dried combination through a sieve to obtain the ferric citrate having a specific surface area greater than 16 sq. m/g, but less than 20 sq. m/g.

In at least one embodiment, the suitable solvent comprises at least one of acetone, methanol, ethanol or isopropyl alcohol, tetrahydrofuran, Acetonitrile, and 1,4-Dioxane.

In another aspect, a pharmaceutical formulation is provided that includes the ferric citrate having a specific surface area greater than 16 sq. m/g, but less than 20 sq. m/g., obtained using the method(s) disclosed herein.

Other aspects will be apparent from the disclosure herein.

### BRIEF DESCRIPTION OF THE DRAWINGS

FIG. 1 illustrates a process for the preparation of ferric citrate according to at least one embodiment of the processes disclosed herein.

For the purposes of illustration, the drawings represent preferred embodiments, it being understood that the invention is not limited to the embodiments shown therein.

### DESCRIPTION

Embodiments of the present application now will be described more fully hereinafter with reference to the accompanying examples and experiments, in which illustrative embodiments of the invention are shown. The invention(s) disclosed herein, however, may be embodied in many different forms and should not be construed as limited to the embodiments set forth herein.

The terminology used herein is for the purpose of describing particular embodiments only and is not intended to be limiting of the invention. As used herein, the singular forms “a”, “an” and “the” are intended to include the plural forms as well, unless the context clearly indicates otherwise. Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

All percentages and ratios used herein are by weight of the total composition and all measurements made are at 25° C. and normal pressure unless otherwise designated. All temperatures are in degrees Celsius unless specified otherwise.

As used herein, “comprising” means the elements recited, or their equivalent in structure or function, plus any other element or elements which are not recited. The terms “having” and “including” are also to be construed as open ended unless the context suggests otherwise.

All ranges recited herein include the endpoints, including those that recite a range “between” two values.

Aspects in the preparation of ferric citrate are quality and the desired property or properties of the end product. Solid oral formulations generally have very stringent requirements regarding certain properties. For example, if certain properties are not uniform throughout the product, the formulation product may not be successful. For ferric citrate, the specific surface area is an important property to meet bioavailability. Surface area is indirectly proportional to the particle size of the molecule. That is, to achieve a higher surface area, particle size is reduced. If the specific surface is not consistent or uniform in a material, bioavailability of the material will be affected. To maintain uniformity of the formulation for bioavailability, processes have been developed and are provided herein which help achieve the specific surface area, preferably greater than 16 sq. m/gm consistently or uniformly in the active pharmaceutical ingredient (API).

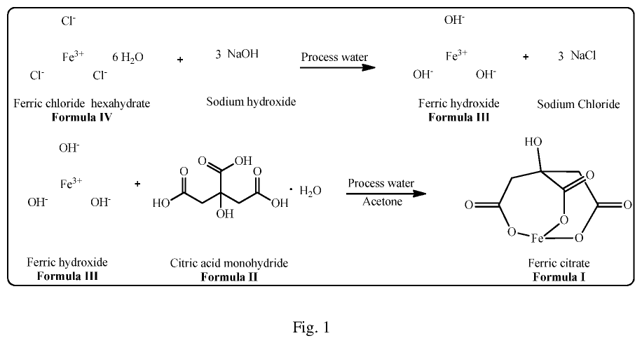

In one of the aspect, the present application provides improved process(es) for the preparation of ferric citrate that includes the steps of a) Substitution reaction of ferric chloride of formula IV with suitable base to get ferric hydroxide of formula III, and b) Reaction of ferric hydroxide with citric acid of formula II to get Ferric citrate of formula I. A schematic representation of improved process is depicted in FIG. 1.

Substitution reaction in step a) preferably includes reaction of ferric chloride with sodium hydroxide by using water as solvent to get ferric hydroxide of formula III. In particular, the ferric chloride is selected in ferric chloride hexahydrate form. Suitable reagents for use in step a) includes, but is not limited to, sodium hydroxide, potassium hydroxide, lithium hydroxide, calcium hydroxide, caesium hydroxide, potassium carbonate, sodium carbonate, potassium bicarbonate, sodium bicarbonate, etc., or a combination thereof.

A suitable temperature for the reaction of step a, may be about 0° to about 40° C., preferably between 20° C. to 30° C., or any other suitable temperatures. The reaction may be carried out for any desired time period ranging from about 30 minutes to about 24 hours or longer.

Reaction in step b) preferably involves the treatment of ferric hydroxide of formula III with citric acid of formula II in water to get ferric citrate of formula I.

Citric acid in step b) is selected from citric acid monohydrate or anhydrous, or any available form of citric acid.

A suitable temperature for the reaction of step b), may be about 10° to about 140° C., preferably between 80° C. to 120° C., or any other suitable temperatures. Preferably, suitable temperature is between 100° C. to 110° C. The reaction may be carried out for any desired time period ranging from about 30 minutes to about 24 hours or longer.

Ferric citrate herein obtained is not isolated as a solid, rather it is kept in liquid state. After the reaction is over, the reaction mixture may be filtered to remove foreign particles to get clear filtrate. Ferric citrate having specific surface area greater than 16 sq. m/g is then obtained by processing the liquid filtrate ml of ferric citrate.

In another aspect of the present application, improved processes for the isolation of ferric citrate from liquid filtrate ml are provided. Several processes have been developed to get ferric citrate having specific surface area greater than 16 sq. m/gm consistently.

In one embodiment, the process involves the initial preparation of ferric citrate having specific surface area greater than 20 sq. m/g. Ferric citrate having specific surface area less than 20 sq. m/g., but greater than 16 sq. m/g may then be obtained from the initial preparation.

In this embodiment, the process of obtaining the initial preparation involves step(s) of:
I. Dumping a suitable solvent into the clear filtrate ml to get solid material, followed by slurring the obtained material in solvent, filtering, and drying the material.
II. Passing the dried material through a sieve to get fine powder having surface area greater than 20 sq. m/g.

Ferric citrate obtained has a particle size greater than required for pharmaceutical dosage form. Particle size distribution should be specific, otherwise bioavailability of ferric citrate will be affected. This leads to preparation of ferric citrate having specific surface area greater than 16 sq. m/g, but less than 20 sq. m/g.

The process to get ferric citrate having specific surface area (SSA) greater than 16 sq. m/g involves one or more of the step(s) of:
I. Ferric citrate (having Specific surface area (SSA) greater than 20 sq. m/g) is sprayed with water,
II. Optionally ferric citrate (having surface area greater than 20 sq. m/g) is charged in solvent alone or mixture of solvent in presence of water, and/or.
III. Mixing, sifting and/or shifting, blending, and drying material to get material having the desired specific surface area and particle size distribution (PSD).

In one of the processes (Example 1), acetone may be dumped into filtrate ml to get the solid material. The material may then be filtered and washed with acetone. Solid material may then be stirred with acetone and filtered to get the material. Solid material is then dried under vacuum. Dried material may then be passed through a sieve to get ferric citrate material (i) having specific surface area greater than 20 sq. m/g. The improve process is provided to get this ferric citrate into one having specific surface area in between 16-20 sq. m/g.

Ferric citrate (i) obtained in above step may be sprayed with 10% water (based upon specific surface area). After properly mixing, shifting, blending, and drying, ferric citrate is obtained which has the specific surface area greater than 16 sq. m/g.

Water quantity for spraying in this step depends on the initial specific surface area. Quantity for spraying is not limited to from 5% w/w to 50% w/w of ferric citrate.

Another process provided (Example 2) involves a charging of ferric citrate (i) into suitable solvent or mixture of solvent in presence of water. The reaction mass is then stirred for few minutes to few hours for get uniform mixing. The material is then filtered and washed with suitable solvent to get ferric citrate having surface area greater than 16 sq. m/g. & less than 20 sq. m/g

Suitable solvent in this step is not limited to water, acetone, methanol, isopropyl alcohol and tetrahydrofuran or any combination thereof.

One of the other processes provided (Example 3) involves dividing the filtrate ml into two equal parts. One filtrate part is dumped with suitable solvent in 2-3 hours to several hrs. addition while another filtrate part is dumped with suitable solvent in one lot. Both lots are then mixed and slurry into suitable solvent to get ferric citrate having specific surface area greater than 16 sq. m/g.

Suitable solvent for dumping is selected from acetone, methanol, ethanol or isopropyl alcohol, tetrahydrofuran, Acetonitrile, 1,4-Dioxane, etc. or any other suitable organic solvent or combination of solvents.

Ferric citrate obtained by this process may then be further passes through sieve to get material having desired specific surface area. Sieve can be selected from any range like 75 microns to 420 microns. Preferably, the material is passed through 75-micron sieve, with or without the mixing, shifting, and/or blending techniques discussed herein.

The Isolation of pure intermediate can be done by decantation, centrifugation, gravity filtration, suction filtration and the like. Drying can be carried out in a tray dryer, vacuum oven, air oven, cone vacuum dryer, rotary vacuum dryer, fluidized bed dryer, spin flash dryer, flash dryer, or the like. The drying can be carried out less than about 60° C., less than about 40° C., less than about 30° C., less than about 20° C., or any other suitable temperatures; at atmospheric pressure or under a reduced pressure; as long as the crystalline intermediate is not degraded in its quality. The drying can be carried out for any desired times until the required product quality is achieved. Preferably, drying is done at 30-40° C. Suitable time for drying can vary from few minutes to several hours for example from about 30 minutes to about 24 or more hours.

Dissolution profile for any solid oral formulation depends upon the particle size and specific surface area. The Ferric citrate synthesize by this route have specific surface area above 16 sq. m/g which helps it to match dissolution profile.

In another embodiment of the present application provides pharmaceutical formulations that include ferric citrate obtained as disclosed herein having specific surface area more than 16 sq. m/gm with one or more pharmaceutically acceptable excipients. The excipients of the present application may be formulated as: solid oral dosage forms such as, but not limited to, powders, granules, pellets, tablets, and capsules. Formulations may be in the forms of immediate release, delayed release, or modified release. This application also presents the use of this Ferric citrate composition in the treatment of hyperphosphatemia and metabolic acidosis.

Other advantage of this route is at least one of: chemical purity, stability—such as storage stability, stability to dehydration, stability to polymorphic conversion, flowability, solubility, morphology or crystal habit, low hygroscopicity and low content of residual solvents.

Certain specific aspects and embodiments of the present application will be explained in more detail with reference to the following examples, which are provided only for purposes of illustration and should not be construed as limiting the scope of the present application in any manner

### Experimental Section

### Method A:

### Preparation of Ferric Hydroxide (Step A):

Ferric chloride hexahydrate (100 g) was dissolved in process water (200 ml) to get clear solution at room temperature (RT). The reaction mass cooled to 0-10° C. Sodium hydroxide solution (45.13 gm in 200 ml water) was prepared and cooled to 0-10° C. Sodium hydroxide solution was charged into above ferric chloride solution by maintaining temperature of reaction mass 0-15° C. within 2-3 hrs. pH of the solution was adjusted to 7.5-8.5 at or below 30° C. and maintained for 30 minutes. Reaction mass was settled for minimum 3.0 hrs. Supernatant water was decanted after solid settled.

Process water (1000 ml) was charged and stirred for 30-45 min. The reaction mas was settled for minimum 3.0 hrs. Then supernatant water was decanted after the solid settled (same operation repeated for 4 more times). The solid was filtered and washed with water (100 X 5 times) to get ferric hydroxide.

Preparation of Ferric Citrate (Step B):

Above solid was charged in process water (250 ml) to make slurry mass & passed through 1mm sieve to brake agglomerate. Citric acid monohydrate (81.63 g) was charged in above solution and stirred to get a slurry mass, reaction mass temperature raised to 80-90° C. Reaction mixture was stirred for 1.0 hours at 80-90° C. Citric acid monohydrate (3.88 g) was charged and further maintained for 1-2 hrs.at 80-90° C. The reaction mass was cooled to 30-40° C. and the reaction mass filtered through cartriage filter to get clear solution.

Preparation of Ferric citrate having specific surface area above 20 m²/g (step c):

The clear filtrate ml was charged to assembly and then acetone was dumped in one lot (2.5 V wrt filtrate). Filtered the solid and then washed with acetone (150 ml). Solid was charged into acetone (1 V wrt filtrate ml) and stirred for 5-10 min. Filtered the solid and washed with acetone (150 ml). Solid was unloaded and dried at 30-40° C. in VTD for 1-2 hrs. Solid was then passed through 20 mesh sieve to get fine powder. The material was then dried under vacuum at 30-40° C. for 10-12 hrs. Material obtained through this process has specific surface area above 20 m²/g.

Procedure to get specific surface area 16-20 m²/g from more than 20 m²/g (step d):

Example-1: Above solid having specific surface area more than 20 m²/g was sprayed with 10% w/w water and the material was mixed well to ensure homogeneity. Shifting and blending was performed to get uniformity. The material was evenly sprayed in trays and then dried at 30-40° C. to achieve water content, SSA, and PSD.

Example-2: The above solid having specific surface area more than 20 m²/g was charged to acetone: water (90:10 v/v) and stirred for 1 hour. The reaction mass was filtered and washed with Acetone. Solid was dried at 30-40° C. to achieve water content, SSA & PSD.

### Method B:

### Preparation of Ferric Hydroxide (Step A):

Ferric chloride hexahydrate (100 g) was dissolved in process water (250 ml) to get clear solution at RT. Sodium hydroxide solution (45.13 gm in 250 ml water) was charged in above reaction mass to get the pH of the solution to greater than 7 at or below 40° C. Reaction mass was maintained for 30 minutes. Filtered the solid & washed with water (100 X 5 times). Precipitated product is again treated with water (250 ml) to remove impurities. Solid is filtered and washed with water (100×5 times) to get ferric hydroxide.

Preparation of Ferric Citrate (Step B):

The above solid was dissolved in process water (250 ml). Citric acid monohydrate (81.63 g) was dissolved in the above solution and temperature was raised to 90-110° C. The reaction mixture was stirred for 1-2 hours at 90-110° C. Citric acid monohydrate (3.88 g) was charged and further maintained for 1-2 hrs. Once the reaction was complete, reaction mass was cooled to 25-30° C. Reaction mass was filtered through celite bed (100 g) and then passed through micron filter paper (0.45 micron).

Preparation of Ferric citrate having specific surface area above 16 sq. m/g.

Example-3: Above filtrate ml is divided in two equal parts.

One lot is taken in a round bottom flask (RBF). Acetone (625 ml) was charged into it within 2-3 hours. Solid was filtered and washed with Acetone (100 ml) (Lot-I wet cake)

Second lot was charged in another RBF and then acetone was dumped in one lot (625 ml) into it. The reaction mass was stirred for 1 hour, solid filtered, and washed with acetone (100 ml). Solid was taken into acetone (500 ml) and stirred for 1-2 hours to get complete powder.

Lot-I wet cake was charged into this slurry and maintained for 1.0-2.0 hr. Solid was filtered and washed with acetone (100 ml×3). Solid was unloaded and dried at 30-40° C. in ATD/VTD for 10-12 hrs. Solid was micronized by passing through sieve (BSS 200/75 micron). Final material was dried under vacuum at 30-40° C. for 10-12 hrs. Material obtained through this process is having specific surface area above 16 sq. m/g.

While the foregoing invention has been described in some detail for purposes of clarity and understanding, it will be appreciated by one skilled in the art, from a reading of the disclosure, that various changes in form and detail can be made without departing from the true scope of the invention.

What is claimed is:

1. A method for obtaining ferric citrate comprising:
reacting ferric chloride with a suitable base to obtain ferric hydroxide;
reacting the ferric hydroxide with citric acid to obtain ferric citrate; and
processing the ferric citrate to obtain ferric citrate having a specific surface area greater than 16 sq. m/g, but less than 20 sq. m/g.

2. The method of claim 1, wherein the ferric chloride is reacted with a suitable base in the presence of water, and wherein the suitable base comprises one or more of: sodium hydroxide, potassium hydroxide, lithium hydroxide, calcium hydroxide, caesium hydroxide, potassium carbonate, sodium carbonate, potassium bicarbonate, and sodium bicarbonate.

3. The method of claim 1, wherein the ferric chloride is ferric chloride formula IV.

4. The method of claim 1, wherein the ferric hydroxide is ferric hydroxide formula III.

5. The method of claim 1, wherein the citric acid is citric acid formula II.

6. The method of claim 1, wherein the ferric citrate is ferric citrate formula I.

7. The method of claim 1, wherein the ferric citrate obtained is in liquid form.

8. The method of claim 7, wherein processing the ferric citrate comprises filtering the liquid ferric citrate to obtain liquid filtrate ml of ferric citrate.

9. The method of claim 7, comprising isolating ferric citrate from the liquid filtrate ml of ferric citrate.

10. The method of claim 9, wherein isolating ferric citrate comprises adding a suitable solvent to the liquid filtrate ml of ferric citrate, slurring resulting solid material in the solvent, and drying the solid material.

11. The method of claim 10, wherein isolating ferric citrate further comprises passing dried ferric citrate through a sieve to obtain ferric citrate powder having surface area greater than 20 sq. m/g.

12. The method of claim 11, wherein isolating the ferric citrate further comprises spraying the ferric citrate powder with water, blending the mixture, drying the mixture, and passing the dried mixture through a sieve to obtain the ferric citrate having a specific surface area greater than 16 sq. m/g, but less than 20 sq. m/g.

13. The method of claim 11, wherein isolating the ferric citrate further comprises charging the dried ferric citrate into a solvent, blending, the mixture, drying the mixture, and passing the dried mixture through a sieve to obtain the ferric citrate having a specific surface area greater than 16 sq. m/g, but less than 20 sq. m/g.

14. The method of claim 11, wherein isolating the ferric citrate further comprises charging the ferric citrate powder with a solvent, stirring the reaction mass, and washing the reaction mass with the solvent.

15. The method of claim 14, wherein the solvent comprises at least one of: acetone, methanol, isopropyl alcohol and tetrahydrofuran.

16. The method of claim 11, wherein isolating the ferric citrate further comprises dividing the liquid filtrate ml of ferric citrate into at least two equal lots; charging each lot of filtrate with a suitable solvent, mixing each lot, creating a slurry comprising the at least two lots, drying the combined lots, and passing the dried combination through a sieve to obtain the ferric citrate having a specific surface area greater than 16 sq. m/g, but less than 20 sq. m/g.

17. The method of claim 16, wherein the suitable solvent comprises at least one of acetone, methanol, ethanol or isopropyl alcohol, tetrahydrofuran, Acetonitrile, and 1,4-Dioxane.

18. A pharmaceutical formulation comprising the ferric citrate having a specific surface area greater than 16 sq. m/g, but less than 20 sq. m/g. obtained using the method of claim 1.
